(19) United States
(12) Patent Application Publication
(10) Pub. No.: US 2014/0127178 A1
(43) Pub. Date: May 8, 2014

(54) METHODS AND COMPOSITIONS TO REDUCE ANIMAL WASTE PARAMETERS

(71) Applicant: Michael Athanasius Olde Dubbelink, Oldenzaal (NL)

(72) Inventor: Michael Athanasius Olde Dubbelink, Oldenzaal (NL)

(21) Appl. No.: 14/155,921

(22) Filed: Jan. 15, 2014

Related U.S. Application Data
(63) Continuation-in-part of application No. PCT/EP2012/063925, filed on Jul. 16, 2012.
(60) Provisional application No. 61/508,271, filed on Jul. 15, 2011.

(30) Foreign Application Priority Data
Jul. 15, 2011 (NL) 1038953
Dec. 12, 2011 (GB) 1121275.0

(51) Int. Cl. A61K 35/74

(52) U.S. Cl. CPC A61K 35/74
USPC 424/93.462; 424/93.46

(57) ABSTRACT

The invention relates to the use of a microbial composition for the simultaneous reduction of important animal waste parameters, such as ammonia emission, the dry matter content, and amount of nitrogen, the amount of ammonium, and the amount of phosphorus present in the animal waste. It further relates to a method for the simultaneous reduction of important animal waste parameters, such as ammonia emission, the dry matter content, and amount of nitrogen, the amount of ammonium, and the amount of phosphorus present in the animal waste; comprising administering a microbial composition to an animal.

### CROSS-REFERENCE TO RELATED APPLICATIONS

This application is filed under 35 U.S.C. §111(a) as a continuation-in-part of International Patent Application No. PCT/EP2012/063925, filed Jul. 16, 2012, which international application designates the United States, and which claims the benefit of Great Britain Application No. 1121275.0 filed Dec. 12, 2011, Netherlands Application No. 1038953 filed Jul. 15, 2011, and U.S. Provisional Application No. 61/508,271 filed Jul. 15, 2011.

The invention relates to the use of a microbial composition consisting of Bacillus amyloliquefaciens, preferably in combination with Bacillus subtilis, Bacillus licheniformis, and Bacillus pumilus for the simultaneous reduction of important animal waste parameters, such as ammonia emission, the dry matter content, and amount of nitrogen, the amount of ammonium, and the amount of phosphorus present in the animal waste. It further relates to a method for the simultaneous reduction of important animal waste parameters, such as ammonia emission, the dry matter content, and amount of nitrogen, the amount of ammonium, and the amount of phosphorus present in the animal waste; comprising administering such a microbial composition to an animal.

### BACKGROUND OF THE INVENTION

Intensive animal agriculture generates high volumes of animal waste and thereby has a strong environmental and economical impact. Several countries introduced legislature regarding manure production, storage and disposal. For example, the Netherlands implemented the Fertilizer Law in 1986, which states that farmers are not allowed to increase the manure production above certain quotas. Above this limit, a manure producer is charged a fee. Since then, similar legislations have been implemented in other countries as well. In general, quotas are expressed in amounts of ammonium (NH4-N), total nitrogen (N) and/or total phosphorus (P2O5). Excessive excretion of these nutrients is implicated in environmental pollution, such as ammonia emission (NH3), surface water pollution, and nitrate and phosphorus leaching. In addition, ammonia emission has a negative effect on animal growth and health. Furthermore, a high dry matter content of manure increases the cost of handling and disposal.

The need for strategies to decrease animal waste production, ammonia, nitrogen and phosphorus content in animal waste, and ammonia emission in animal agriculture is therefore of critical importance for environmental as well as economical reasons. In countries with legislation that imposes quotas on farmers, decreased manure, ammonium, nitrogen and phosphorus production per animal leads to larger numbers of animals that can be housed and has therefore major economical consequences.

Current possibilities to decrease these animal waste parameters in agriculture are limited and generally involve nutritional management strategies. Nutritional management methods include changes in the feed composition, addition of enzymes to the feed to increase feed utilisation and careful feeding dosage and timing. However, nutritional management is often complex and generally results in higher costs due to additional farm infrastructure, higher transport costs and expensive feed additives. Even with these strategies, effects on animal waste, ammonium, nitrogen and/or phosphorus production and ammonia emission are, in general, limited. Furthermore, the currently available strategies generally lead to a reduction of only one of these parameters.

For example, WO2009007192 provides a bacillus spores composition that secretes high levels of phytase and that is to be added to animal (e.g. pig) feed. Phytase is frequently used in monogastric animal feed to enhance phytate digestion, thereby lowering phosphorus content in animal waste. The bacillus cells of this application are mutated and selected for their rapid germination and outgrowth in bile salt (simulated gut environment) and their high secretion of phytase. Since WO2009007192 does not include experimental data on possible phosphorus reduction in animal waste after administering this bacillus spores composition, it is not known if and by how much the phosphorus content is reduced. Furthermore, the composition only targets the phosphorus uptake by the animal and is not expected to influence the other important animal waste parameters, i.e. nitrogen content, ammonium content, ammonia emission, and animal waste production.

U.S. Pat. No. 6,410,305 on the other hand, focuses on the treatment of animal waste. The treatment comprises two steps: a) administering a bacterial mixture to an animal to promote organic digestion, and b) adding an inoculum comprising sulfide-utilizing bacteria to waste produced by the animal. Example 4 from this application describes an experiment that focuses on step a). Inclusion of the bacterial mixture, several lytic enzymes, Yucca schidigera extracts, extruded wheat and limestone in the swine diet leads to a reductions of 43% of total solids after 90 days, compared to a reduction of 22% of total solids in the control group. Although the total solids are reduced, this method requires complex feed additives containing not only a bacterial mixture, but also several enzymes, plant extracts, pretreatment of feed components and limestone. In addition, this method leads only to the reduction of the total solids and not to a reduction in the total nitrogen, total ammonium and/or total phosphorus content.

Maxwell et al. (1983) found that addition of two commercial probiotics in the feed of pigs both led to a simultaneous reduction of ammonia production and increase of nitrogen and dry matter digestibility. However, the observed differences are rather small (less than 6% for nitrogen and dry matter digestibility).

The sole example in Canadian patent application CA2555516 shows that a pig feed composition further comprising dehydrated and fermented Bacillus licheniformis, Bacillus subtilis and Aspergillus niger as micro-organisms results in a liquefaction of the solid fraction in the manure, a reduction in the proportion of nitrogen and anhydride sulfide gases, with a reduction of the phosphorus in the manure.

It further suggests that similar results may be expected in case other probiotic bacteria are used including the Bacillus species Bacillus subtilis, Bacillus latereosporus, Bacillus licheniformis, Bacillus pumilis, Bacillus megaterium, and Bacillus amyloliquifaciens.

However, results of other groups show that the observations done with one composition of micro-organisms can not simply be extrapolated to any other combination of micro-organisms. In fact, several other groups showed findings to contrary, in that the effects of probiotic Bacillus mixtures that were added to the diet of pigs, had no effects on dry matter and/or nitrogen digestibility at all. Chen et al. (2006) observed that supplementation of a Bacillus-based mixture containing B. subtilis, Bacillus coagulans and Lactobacillus acidophilus did not affect dry matter or nitrogen digestibility. A mixture of B. subtilis and B. licheniformis also does not affect dry matter or nitrogen digestibility (Kornegay and Risley, 1996, Wang et al., 2008), although it reduces ammonia emission from slurry (Wang et al., 2008). Furthermore, a mixture of B. subtilis, B. licheniformis and Bacillus pumilus also did not improve dry matter or nitrogen digestibility (Kim et al., 1993, Kornegay and Risley, 1996).

Therefore, an objective of the present invention is to provide a method to decrease simultaneously multiple important animal waste parameters, such as the total solids content of the animal waste, the total nitrogen production, the total ammonium and ammonia production, and the total phosphorus production by an animal through the administration of a dietary microbial composition to the animal, with in particular an effect on dry matter and/or nitrogen digestibility; in particular a composition comprising a combination of the Bacillus species; Bacillus amyloliquefaciens, Bacillus subtilis, Bacillus licheniformis, and Bacillus pumilus as microbial component.

These and other aspects of the present invention will become apparent to those skilled in the art after a reading of the following description of the preferred embodiment.

### SUMMARY OF THE INVENTION

It has surprisingly been found that a microbial composition comprising Bacillus amyloliquefaciens; in particular in combination with the Bacillus strains, Bacillus subtilis, Bacillus licheniformis, and Bacillus pumilus can be used to reduce multiple important animal waste parameters simultaneously, which has considerable environmental and economical advantages. It is thus a first object of the present invention to provide the use of a microbial composition to reduce at least two of the following parameters of animal waste:
the dry matter content of the animal waste,
the ammonia emission,
the total amount of ammonium present in the animal waste,
the total amount of nitrogen present in the animal waste, and
the total amount of phosphorus present in the animal waste;
   characterized in that the microbial composition comprises Bacillus amyloliquefaciens, in particular in combination with the Bacillus strains, Bacillus subtilis, Bacillus licheniformis, and Bacillus pumilus.

Furthermore, it has been found that a microbial composition comprising B. amyloliquefaciens, in particular in combination with the Bacillus strains, Bacillus subtilis, Bacillus licheniformis, and Bacillus pumilus; enhances dry matter digestibility and mineral absorption. Several minerals are currently added to animal feed to reach the nutritional requirements. Nonetheless, most of these minerals are already present in the animal feed, but in a non-digestible form. For example, phytate is the principal storage form of phosphorus in many plant tissues, especially in grains. Since most nonruminant animals cannot digest it, phosphate is often added to animal feed. Due to the greatly increased prices of phosphate, this has a significant impact on animal feed costs. Furthermore, the undigested phytate contributes to a higher phosphate content of manure. It has surprisingly been found that the microbial composition of the invention increases phosphate digestibility. Therefore, administration of the microbial composition to an animal reduces the amount of phosphate that needs to be added to the animal feed. Therefore, the invention also provides economical and environmental benefits regarding the animal feed production.

It is thus a second object of the invention to provide the use of a microbial composition to enhance dry matter digestibility and/or mineral absorption, characterized in that the microbial composition comprises Bacillus amyloliquefaciens, in particular in combination with the Bacillus strains, Bacillus subtilis, Bacillus licheniformis, and Bacillus pumilus. It is accordingly also an object of the invention to provide such a microbial composition that allows to administer to an animal a feed with a reduced mineral content, in particular a reduced nitrogen and/or phosphorus/phosphate content, more in particular a reduced phosphorus/phosphate content.

Evidently, the present invention also provides a microbial composition comprising one or more of the Bacillus strains mentioned herein.

### DETAILED DESCRIPTION

As described previously, it is thus a first object of the present invention to provide the use of a microbial composition to reduce at least two of the following parameters of animal waste:
the dry matter content of the animal waste,
the ammonia emission,
the total amount of ammonium present in the animal waste,
the total amount of nitrogen present in the animal waste, and
the total amount of phosphorus present in the animal waste;
   characterized in that the microbial composition comprises Bacillus amyloliquefaciens, in particular in combination with the Bacillus strains, Bacillus subtilis, Bacillus licheniformis, and Bacillus pumilus.

In a more particular embodiment the Bacillus species used in the microbial compositions throughout this application are Bacillus amyloliquefaciens strain ATCC Nr 21415; Bacillus subtilis strain ATCC Nr 27505; Bacillus licheniformis strains ATCC Nr 10716; and Bacillus pumilis strain NRRL B-4064. Each of said strains has also been deposited with the Deutsche Sammlung von Mikro-organismen and Zellkulturen GmbH (DSMZ), Inhoffenstraβe 7B, 38124 Braunschweig, Germany on Jul. 12, 2012 wherein;
Bacillus amyloliquefaciens strain ATCC Nr 21415 has been annotated as Bacillus amyloliquefaciens NBS 48420 and received the accession number DSM 26163;
Bacillus subtilis strain ATCC Nr 27505 has been annotated as Bacillus subtilis NBS 13923 and received the accession number DSM 26162;
Bacillus licheniformis strain ATCC Nr 10716 has been annotated as Bacillus licheniformis NBS 72100 and received the accession number DSM 62164; and
Bacillus pumilus strain NRRL B-4064 has been annotated as Bacillus pumilis NBS 93064 and received the accession number DSM 26165.

Animal waste comprises all liquids, solids and gases that are excreted by the animal; in particular animal waste comprises urine, faeces, and gases from belching and flatulence.

In a further embodiment, the present invention provides the use of a microbial composition as characterized herein, to reduce the dry matter content of the animal waste and at least one of the following parameters of the animal waste:
the ammonia emission,
the total amount of ammonium present in the animal waste,
the total amount of nitrogen present in the animal waste, and
the total amount of phosphorus present in the animal waste.

In an even further embodiment, the present invention provides the use of said microbial composition to reduce the dry matter content of the animal waste and at least two of the following parameters of the animal waste:
the ammonia emission,
the total amount of ammonium present in the animal waste,
the total amount of nitrogen present in the animal waste, and
the total amount of phosphorus present in the animal waste.

In an even further embodiment, the present invention provides the use of a microbial composition as characterized herein, to reduce the dry matter content of the animal waste and at least three of the following parameters of the animal waste:
the ammonia emission,
the total amount of ammonium present in the animal waste,
the total amount of nitrogen present in the animal waste, and
the total amount of phosphorus present in the animal waste.

In particular, the present invention provides the use of a microbial composition to reduce the dry matter content of the animal waste, the ammonia emission, the total amount of ammonium present in the animal waste, the total amount of nitrogen present in the animal waste, and the total amount of phosphorus present in the animal waste.

As described previously, it is a second object of the present invention to provide the use of a microbial composition to enhance dry matter digestibility and/or mineral absorption, characterized in that the microbial composition comprises Bacillus amyloliquefaciens, in particular in combination with the Bacillus strains, Bacillus subtilis, Bacillus licheniformis, and Bacillus pumilus. In a particular embodiment, said mineral is one or more minerals selected from the group consisting of nitrogen, phosphorus, magnesium, potassium, and calcium; more in particular from the group consisting of nitrogen and phosphorus. In another embodiment, said mineral is phosphorus.

In a further embodiment, the use of a microbial composition according to the invention comprises administering said composition to an animal. In a particular embodiment, said composition is administered to said animal by adding it to the diet. In another particular embodiment, said composition is administered by nebulization. In yet another particular embodiment, said composition is administered by the combination of nebulization and adding it to the diet.

In principle, said animal can be any animal, but typically, said animal is a farm animal or a pet. In particular, said farm animal is selected from the group comprising a pig, a sheep, a goat, a cow, a horse, a chicken, a duck, a goose, a turkey, and a rabbit. More in particular said farm animal is a monogastric animal, such as a horse, a rabbit or a pig. Even more in particular said farm animal is a pig.

In a further embodiment, the microbial composition for use in the methods of the present invention comprises bacteria, including Bacillus amyloliquefaciens. In an even further embodiment, said microbial composition comprises Bacillus amyloliquefaciens and one or more other species of Bacillus. In a particular embodiment, the microbial component of said composition consists of one or more species of Bacillus, including Bacillus amyloliquefaciens.

When Bacillus species are used in the microbial composition according to the present invention, said other species are particularly selected from Bacillus megaterium, Bacillus subtilis, Bacillus licheniformis, and Bacillus pumilus. In particular, said Bacillus species consist of Bacillus amyloliquefaciens, Bacillus subtilis, Bacillus licheniformis, and Bacillus pumilus.

In another embodiment, said Bacillus species are present in the microbial composition in roughly equal amounts. Therefore, said Bacillus species may consist of about 25% Bacillus amyloliquefaciens, about 25% Bacillus subtilis, about 25% Bacillus licheniformis, and about 25% Bacillus pumilus. In particular, the microbial composition of the invention comprises Bacillus amyloliquefaciens, preferably in combination with Bacillus subtilis, Bacillus licheniformis, and Bacillus pumilus.

In a particular embodiment of the present invention the Bacillus species used in the microbial compositions for use in the methods of the present invention are selected from one or more of; the Bacillus amyloliquefaciens strains Bacillus amyloliquefaciens with ATCC Nr 21415 or NCBI strain FZB42; the Bacillus subtilis strains ATCC Nr 27505 or ATCC Nr 6051; the Bacillus licheniformis strains ATCC Nr 10716 or ATCC Nr 14580; and the Bacillus pumilus strain of the ARS culture collection with accession Nr NRRL B-4064. In a more particular embodiment the Bacillus species used in the microbial compositions are Bacillus amyloliquefaciens strain ATCC Nr 21415 (also deposited with DSMZ supra—Nr DSM 26163); Bacillus subtilis strain ATCC Nr 27505 (also deposited with DSMZ supra—Nr DSM 26162); Bacillus licheniformis strains ATCC Nr 10716 (also deposited with DSMZ supra—Nr DSM 26164); and Bacillus pumilis strain NRRL B-4064 (also deposited with DSMZ supra—Nr DSM 26165).

As known by the skilled person, microbial compositions are generally made by fermentation. Microbial cells may be present as vegetative cells or spores. When the composition comprises spores, the obtained spore cells are generally concentrated, dried, mixed with a carrier and packed into a suitable container.

It is a further objective of the present invention to provide a method to reduce at least two of the following parameters of the animal waste:
the dry matter content of the animal waste,
the ammonia emission,
the total amount of ammonium present in the animal waste,
the total amount of nitrogen present in the animal waste, and
the total amount of phosphorus present in the animal waste;
   said method comprising administering a microbial composition according to the invention to an animal; in particular a monogastric animal, such as a horse, a rabbit or a pig; even more in particular a pig.

In another embodiment the present invention provides a method to reduce the dry matter content of the animal waste and at least one of the following parameters of the animal waste:
the ammonia emission,
the total amount of ammonium present in the animal waste,
the total amount of nitrogen present in the animal waste, and
the total amount of phosphorus present in the animal waste;
   said method comprising administering a microbial composition according to the invention to an animal; in particular a monogastric animal, such as a horse, a rabbit or a pig; even more in particular a pig.

In another embodiment the present invention provides a method to reduce the dry matter content of the animal waste and at least two of the following parameters of the animal waste:
the ammonia emission,
the total amount of ammonium present in the animal waste,
the total amount of nitrogen present in the animal waste, and
the total amount of phosphorus present in the animal waste;
   said method comprising administering a microbial composition according to the invention to an animal; in particular a monogastric animal, such as a horse, a rabbit or a pig; even more in particular a pig.

In another embodiment the present invention provides a method to reduce the dry matter content of the animal waste and at least three of the following parameters of the animal waste:
the ammonia emission,
the total amount of ammonium present in the animal waste,
the total amount of nitrogen present in the animal waste, and
the total amount of phosphorus present in the animal waste;
   said method comprising administering a microbial composition according to the invention to an animal; in particular a monogastric animal, such as a horse, a rabbit or a pig; even more in particular a pig.

In a further embodiment the present invention provides a method to reduce the dry matter content of animal waste, the ammonia emission, the total amount of ammonium present in the animal waste, the total amount of nitrogen present in the animal waste, and the total amount of phosphorus present in the animal waste; comprising administering a microbial composition according to the invention to an animal; in particular a monogastric animal, such as a horse, a rabbit or a pig; even more in particular a pig.

The microbial composition used in any one of the aforementioned methods is administered to the animal as defined hereinbefore.

In analogy with the foregoing uses, the animals in any one of the aforementioned methods can be any animal, but is typically a farm animal or a pet. In particular said farm animal is selected from the group comprising a pig, a sheep, a goat, a cow, a horse, a chicken, a duck, a goose, a turkey, and a rabbit. More in particular said farm animal is a monogastric animal, such as a horse, a rabbit or a pig. Even more in particular said farm animal is a pig.

Said microbial composition in any one of the methods according to the present invention, include each of the embodiments as defined hereinbefore

As mentioned herein, the present invention provides a composition comprising one or more of the Bacillus strains as described herein. In a particular embodiment, the present invention provides a composition comprising Bacillus amyloliquefaciens DSM 26163. Preferably, said composition further comprises one or more bacteria selected from the group consisting of Bacillus subtilis, Bacillus licheniformis, and Bacillus pumilis. In a further embodiment, said composition further comprises one or more bacteria selected from the group consisting of Bacillus subtilis DSM 26162, Bacillus licheniformis DSM 62164, and Bacillus pumilis DSM 26165.

It is another object of the present invention to provide a composition comprising two or more bacterial strains selected from Bacillus amyloliquefaciens DSM 26163, Bacillus subtilis DSM 26162, Bacillus licheniformis DSM 62164, and Bacillus pumilus DSM 26165. In a preferred embodiment, the present invention provides a composition comprising three or more strains selected from said group. In an even more preferred embodiment, the present invention provides a composition comprising Bacillus amyloliquefaciens DSM 26163, Bacillus subtilis DSM 26162, Bacillus licheniformis DSM 62164, and Bacillus pumilus DSM 26165.

Preferably, the microorganisms mentioned herein are present in the composition as spores. Additional microorganisms may be added to the composition of the invention, these may be present as spores or not. Thus, in a particular embodiment, the present invention provides a composition comprising spores of Bacillus amyloliquefaciens DSM 26163.

The compositions of the invention can be administered to an animal, preferably orally. Therefore, the present invention provides a feed additive, comprising the microorganisms of the invention. Evident from the description provided herein, the present invention further provides a method for improving animal waste parameters, comprising administering the feed additive of the invention to a monogastric animal, thereby improving parameters of animal waste excreted by said animal. The method of the invention may further comprise adding the feed additive to feed. The methods of the invention allow an increased uptake of minerals, phosphorus, nitrogen, dry matter, etcetera from said feed.

### EXAMPLES

The invention will be better understood with reference to the following non-limiting examples; the examples are for the purpose of describing a preferred embodiment of the invention and are not intended to limit the invention thereto.

### Example 1

### Effect of Nebulization on Ammonia Emission and on Ammonium, Nitrogen, and Phosphorus Content of Animal Waste

### A. Experimental Setup

Pigs were allocated to two separate compartments, 82 pigs to a control compartment and 123 pigs to a treatment compartment. Pigs in the treatment compartment were handled identically to the pigs in the control compartment, but were administered a supplementary microbial composition through nebulization. The microbial composition consisted of approximately equal amounts of spores from Bacillus amyloliquefaciens DSM 26163, Bacillus subtilis DSM 26162, Bacillus licheniformis DSM 26164, and Bacillus pumilus DSM 26165. This composition was dissolved in water to obtain a solution of approximately 7% (w/v). Every two hours part of the solution was nebulized during 30 seconds. The total amount of nebulized bacteria was 10⁹ colony-forming units (CFU) per animal per day. Pigs in the control and treatment compartment received the same standard pig feed. Until 25 kg, pigs received the following feed: barley, wheat, wheat bran, soy, corn, potato, flax, calcium carbonate, sodium chloride, and calcium sodium phosphate; to which vitamins, minerals, xylanase and phytase were added. After reaching 25 kg, pigs received: soy, wheat, wheat bran, calcium carbonate, monocalcium phosphate, sodium chloride; to which vitamins, minerals, and phytase were added.

The ammonia concentration in the compartments was measured a first time when the animals reached 45 kg and a second time when the animals reached 85 kg. During the ammonia measurements with pigs of 85 kg, the ventilation in the treatment compartment was lower than in the control compartment (45% ventilation rate compared to 60%). After 140 days, when the animals reached slaughter weight, several parameters of the animal waste were determined in an independent testing facility using standard analyses. The dry matter content of the manure was determined according to the procedure described in VDLUFA Bd. II.1, 11.5.1; the total ammonium content (NH4-N) of the manure was determined using the procedure in DIN 38406 (E-51); the total phosphorus content (P2O5) of the manure was determined using the procedure described in ISO 11885:2009; and the total nitrogen content (N) of the manure was determined using the procedure of VDLUFA Bd. II.1, 3.5.2.7.

### B. Results

| | Control | Treatment | Ammonia |
|---|---|---|---|
| Weight of pigs | compartment | compartment | emission |
| (kg) | ammonia (ppm) | ammonia (ppm) | reduction |
| 45 | 33.3 | 26.7 | 19.8% |
| 85 | 49.0 | 23.0 | 53.0% |

Thus, significant reductions of ammonia emissions were observed. Despite the lower ventilation rate in the treatment compartment for the pigs of 85 kg, there was still a large reduction of 53%. In addition, during both experiments, the researchers observed a markedly better smell in the treatment compartments.

Similar experiments were also carried out in which the same microbial composition was sprayed on the floor of the compartment instead of nebulized in the compartment. In these experiments, no reduction of ammonia emissions was observed. This indicates that to be effective, the microbial composition needs to be administered to the animal and not to the housing of the animal.

| | Control | Treatment | Reduction |
|---|---|---|---|
| Manure parameter | (% w/v) | (% w/v) | (%) |
| Dry matter | 8.2 | 7.4 | 9.8 |
| Ammonium (NH4—N) | 0.63 | 0.20 | 68.3 |
| Phosphorus (P2O5) | 0.47 | 0.40 | 14.9 |
| Nitrogen (N) | 0.85 | 0.76 | 10.6 |

### Example 2

### Effect of Dietary Supplementation on Dry Matter, Ammonium, Nitrogen and Phosphorus Content of Animal Waste

### A. Experimental Setup

In this experiment, 144 pigs were treated during 138 days. Pigs were fed the same standard pig diet as in experiment 1. In addition, the pigs received a dietary supplement of spores from Bacillus amyloliquefaciens DSM 26163, Bacillus subtilis DSM 26162, Bacillus licheniformis DSM 62164, and Bacillus pumilus DSM 26165 in approximately equal amounts, mixed with a carrier (lactose). This microbial composition was added to the feed at 4.10⁷ CFU per day per pig. After 138 days, animal waste parameters were determined as in experiment 1; therefore obtained results can also be compared to the control of this experiment.

### B. Results

| | Control | Treatment | Reduction |
|---|---|---|---|
| Manure parameter | (% w/v) | (% w/v) | (%) |
| Dry matter | 8.2 | 5.1 | 37.8 |
| Ammonium (NH4—N) | 0.63 | 0.43 | 31.7 |
| Phosphorus (P2O5) | 0.47 | 0.33 | 29.8 |
| Nitrogen (N) | 0.85 | 0.57 | 32.9 |

### Example 3

### Effect of Spraying and Dietary Supplementation on Dry Matter, Ammonium, Nitrogen and Phosphorus Content of Animal Waste

### A. Experimental Setup

216 piglets of 7 kg received the same standard diet for pigs under 25 kg as described above. Their diet was supplemented with the same microbial composition as in experiment 2; each piglet received 2.10⁷ CFU of this composition per day. In addition, the microbial composition of experiment 1 was sprayed on the floor of the compartment in an amount of 10.10⁹ CFU/m² per week. After 26 days of treatment, animal waste parameters were determined as in experiment 1. These measurements were compared to averages from a database of German piglet waste parameters.

### B. Results

| | Control | Treatment | Reduction |
|---|---|---|---|
| Manure parameter | (% w/v) | (% w/v) | (%) |
| Dry matter | 6.5 | 2.9 | 55 |
| Ammonium (NH4—N) | 0.30 | 0.27 | 10.0 |
| Phosphorus (P2O5) | 0.21 | 0.10 | 51.4 |
| Nitrogen (N) | 0.40 | 0.33 | 16.5 |

1. A composition comprising Bacillus amyloliquefaciens DSM 26163 and one or more strains selected from Bacillus subtilis DSM 26162, Bacillus licheniformis DSM 62164, and Bacillus pumilus DSM 26165.

2. The composition of claim 1 comprising Bacillus amyloliquefaciens DSM 26163, and two or more strains selected from Bacillus subtilis DSM 26162, Bacillus licheniformis DSM 62164, and Bacillus pumilus DSM 26165.

3. The composition of claim 1 comprising Bacillus amyloliquefaciens DSM 26163, Bacillus subtilis DSM 26162, Bacillus licheniformis DSM 62164, and Bacillus pumilus DSM 26165.

4. The composition of claim 3, wherein said Bacillus amyloliquefaciens DSM 26163, Bacillus subtilis DSM 26162, Bacillus licheniformis DSM 62164, and Bacillus pumilus DSM 26165 are present in approximately the same amount.

5. A feed additive comprising the composition of claim 1.

6. A method for improving animal waste parameters comprising administering the composition of claim 1 to a monogastric animal,thereby improving parameters of animal waste excreted by said animal.

7. The method of claim 6, wherein the improvement of animal waste parameters comprises a reduction of the phosphorus content of said animal waste.

8. The method of claim 6, wherein the improvement of animal waste parameters comprises a reduction of the nitrogen content of said animal waste.

9. The method of claim 6, wherein the improvement of animal waste parameters comprises a reduction of ammonia emission from said animal.

10. The method of claim 6, wherein the improvement of animal waste parameters comprises a reduction of:
the dry matter content,
the ammonia emission,
the total amount of ammonium,
the total amount of nitrogen, and
the total amount of phosphorus.

11. The method of claim 6, wherein said composition is administered to the animal by adding it to the feed.

12. The method of claim 6, wherein said composition is administered to the animal by nebulization.

13. The method of claim 6, wherein said composition is administered to the animal by the combination of nebulization and adding it to the feed.

14. The method of claim 6, wherein said monogastric animal is a pig.
